(19) United States
(12) Patent Application Publication
(10) Pub. No.: US 2005/0085522 A1
(43) Pub. Date: Apr. 21, 2005

(54) Alkylphenyliminoimidazolidine derivatives for treating urinary incontinence

(75) Inventors: Pascale Pouzet, Biberach (DE); Franz Esser, Ingelheim (DE); Matthias Hoffmann, Mittelbiberach (DE); Hisato Kitagawa, Osaka (JP); Kenji Sakai, Hyogo (JP); Ikunobu Muramatsu, Yoshida-gun (JP)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/006,375

(22) Filed: Dec. 7, 2004

Related U.S. Application Data
(63) Continuation of application No. 10/058,456, filed on Jan. 28, 2002, now abandoned.
(60) Provisional application No. 60/270,333, filed on Feb. 21, 2001.

(30) Foreign Application Priority Data
Feb. 10, 2001 (DE) 101 06 214.1

(51) Int. Cl. A61K 31/4172; C07D 233/66

(52) U.S. Cl. 514/386; 548/326.5

(57) ABSTRACT

A compound of formula I or of tautomeric formula II 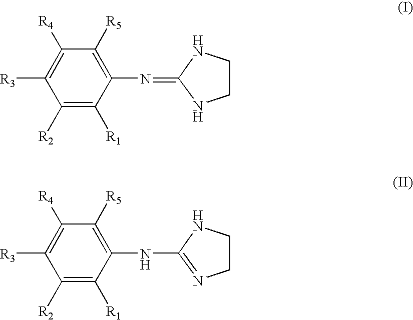
wherein: R₁ and R₅ are each independently H, F, Cl, Br, CH₂F, CHF₂, CF₃, Me, or OMe;
R₂ and R₄ are each independently H, a branched C₃₋₆-alkyl, F, Cl, Br, CH₂F, CHF₂, CF₃ or Me, wherein at least one of the groups R₂ or R₄ is a branched C₃₋₆-alkyl; and
R₃ is H, F, Cl, Br, CH₂F, CHF₂, CF₃ or Me, or a pharmacologically acceptable salt thereof, pharmaceutical compositions containing these compounds, and their use in treating urinary incontinence or a bladder disease.

### RELATED APPLICATIONS

Benefit under 35 U.S.C. § 119(e) of prior provisional application Ser. No. 60/270,333, filed Feb. 21, 2001, is hereby claimed.

### FIELD OF THE INVENTION

The present invention relates to m-alkylphenyliminoimidazolidine derivatives with a new pattern of substituents at the phenyl ring and their use in preparing pharmaceutical compositions, particularly for treating urinary incontinence.

### BACKGROUND OF THE INVENTION

The compounds described within the scope of the present invention belong to the category of m-alkylphenyliminoimidazolidines. Similar compounds are known from the prior art.

WO 96/32939, to which reference is hereby made in its entirety, discloses phenyliminoimidazoles. These include those compounds wherein the phenyl ring contains, inter alia, amino, amido, imido, halo, heteroaryl, cycloalkyl, and alkyl substituents. The compounds described therein count as alpha-1L-agonists and may advantageously be used in this capacity for the treatment of urinary incontinence.

By incontinence is meant an involuntary release of urine, i.e., weakness of the bladder. The various manifestations of urinary incontinence include urge incontinence, reflex incontinence, overflow incontinence and stress incontinence. Among the most common forms of urinary incontinence is stress incontinence. Women, in particular, are affected by this after more or less difficult childbirth. The reason for this is that pregnancy and labour easily lead to a weakening of the pelvic floor. Other causes of incontinence may lie, for example, in damage to the nerves of the pelvic floor, a congenitally short urethra or damage to the sphincter muscle.

It is beneficial to use alpha-1L-agonists in the treatment of urinary incontinence, as they act selectively on the adrenoreceptors of the bladder and thus have a crucial effect on the tonicity of the urethra, without significantly affecting the cardiac circulatory system.

For some time, the possibility of using imidazole derivatives to treat incontinence has been discussed in the prior art. Surprisingly, opinions have been expressed indicating that many imidazole derivatives can counteract weakness of the bladder, while other authors have observed apparently the reverse effect, namely that these substances can give relief in occlusion of the bladder. Still other authors report that these same substances would have no effect at all on bladder function.

Thus, it is reported that alpha 2 agonists such as clonidine would have a positive effect on nocturnal incontinence (Urology, 43 (3) (1994) pp. 324-327). On the other hand, the contrary observation has been made with regard to clonidine, that this substance could even promote incontinence (Clin. Biol. Res. 78 (1981) pp. 101-103). A similar observation is expressed in Jpn. J. Pharmacol. 58 (4) (1992) pp. 339-346. The authors have found that clonidine does not have a clear effect on bladder function, but that phenylethanolamines, such as the phenylephrines, midodrines or ST 1059 which resemble adrenaline, all of which are alpha 1 agonists, do have an effect. EP-A-0 416 841 also relates to the effect of alpha agonists on bladder function. It states that alpha 1 adrenoceptor-blocking substances can be used to treat occlusion of the bladder. The observations according to U.S. Pat. No. 4,226,773 also point in this direction. According to this specification, pyrazolyliminoimidazole derivatives can be used to promote the release of urine. Other alpha 1 adrenergic imidazoles such as thiophenepyrroles, for example, may be used to treat urinary incontinence (EP-A-0 599 697).

These different observations from the prior art lead one to conclude that it has not hitherto been possible to predict the effect of imidazole derivatives on bladder function.

### SUMMARY OF THE INVENTION

Compounds which can be used to treat urinary incontinence not only have to be sufficiently effective but should also have as few side effects as possible. In other words, they should act as selectively as possible on the bladder alone. The unwanted side effects include, inter alia, a negative effect on the cardiac circulatory system. The bioavailability of the substances and their metabolism are of particular importance to particularly effective treatment of urinary incontinence. The bioavailability should be as high as possible and the metabolism should be such that, on the one hand, the substances are not broken down too quickly and, on the other hand, no toxic or other compounds with pharmacological properties which are undesirable in this context are formed.

Therefore, one of the aims of the present invention is to find new alpha 1L-agonists from the category of phenyliminoimidazolidines which act selectively on the bladder without substantially affecting the cardiac circulatory system and have favorable properties with regard to bioavailability or metabolism.

Surprisingly, it has been found that the m-alkylphenyliminoimidazolidines according to the invention achieve the aims of the present invention and are therefore suitable for treating urinary incontinence.

Alkylphenyliminoimidazolidines with branched alkyl groups are known in principle from the prior art.

Thus, for example, WO 92/21349 discloses a number of phenyliminoimidazolidine derivatives for ophthalmological use. DE 1929950 describes, inter alia, 2′-bromo-5′-chloro-4′-tert-butylphenylimini-2-imidazolidine and DE 0116768 describes 2,6-dichloro-4′-tert-butylphenyliminoimidazolidine. EP 0035393 also relates to alkylphenyliminoimidazolidines, albeit not for use in human medicine, but for the production of hens' eggs.

### DETAILED DESCRIPTION OF THE INVENTION

The alkylphenylimino-2-imidazolidine derivatives according to the invention are characterized in that there is a branched C₃-C₆-alkyl group, such as, e.g., isopropyl, isobutyl, tert-butyl, isopentyl, or neopentyl, in at least one of the two possible meta positions to the imino group. Preferably, there is an isopropyl and/or a tert-butyl group. The preferred compounds according to the invention are described by general formula I: 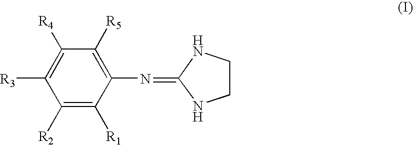
wherein:
R₁ and R₅ independently of one another each denote H, F, Cl, Br, CH₂F, CHF₂, CF₃, Me, or OMe;
R₂ and R₄ independently of one another each denote H, iPr, tert-Bu, F, Cl, Br, CH₂F, CHF₂, CF₃ or Me, while at least one of the groups R₂ or R₄ is iPr or tert-Bu; and
R₃ is H, F, Cl, Br, CH₂F, CHF₂, CF₃, or Me.

Me denotes methyl, CH₂F denotes fluoromethyl, CHF₂ denotes difluoromethyl, CF₃ denotes trifluoromethyl, iPr denotes isopropyl, H denotes hydrogen, F denotes fluorine, Cl denotes chlorine, Br denotes bromine, and tert-Bu denotes tertiary butyl.

If R₅ is OMe, compounds wherein R₂ is tert-Bu are preferred.

Of the compounds specified, the preferred ones are those wherein
R₁ and R₅ independently of one another each denote H, F, Cl, Br, CF₃, Me, or OMe;
R₂ and R₄ independently of one another each denote H, iPr, tert-Bu, or Me, while at least one of the groups R₂ or R₄ is iPr or tert-Bu
R₃ is H, F, Cl, Br, or Me.

Of these, particularly preferred compounds are those wherein
R₁ is H, Cl, Br, or Me;
R₂ is iPr or tert-Bu;
R₃ is H, Br, or Cl;
R₄ is H; and
R₅ is H, Cl, Br, or OMe.

Most preferred are compounds wherein:
R₁ is H or Me;
R₂ is iPr or tert-Bu;
R₃ is H, Cl, or Br;
R₄ is H; and
R₅ is H, Cl, or OMe.

In every case R₂ is preferably iPr or tert-Bu, while R₄ is preferably H. Again, in every case, R₁ is preferably different from OMe.

The compounds represented by formula I may be in tautomeric equilibrium with the alkylanilino-2-imidazoline derivatives of formula II: 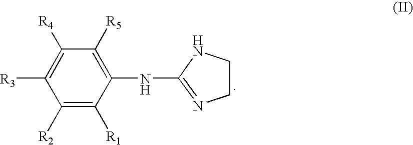
wherein the definitions of the groups R₁, R₂, R₃, R_{4,} and R₅ are identical to those of the abovementioned compounds of formula I including all the preferences listed.

Therefore, the present invention also relates to the compounds coming under general formula II in which the groups R₁, R₂, R₃, R_{4,} and R₅ fall within the scope of the definition given under formula I. The same is true of the preferred ranges mentioned under formula I.

The compounds which come under the scope of the definitions of formulae I and II are equally preferred but independently of one another.

With regard to the nomenclature used in the present invention it should be pointed out that the term “phen-1′-yl-2-imidazolidine” denotes compounds having the following structural element:

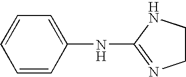

This means that the atoms of the imidazole ring are numbered 1, 2, 3, etc., one nitrogen atom being allocated the number 1 and the other nitrogen atom being allocated the number 3. Accordingly, the imino group is bound to the carbon atom, which is allocated the number 2. The atoms of the phenyl ring are numbered 1′, 2′, 3′, etc., while the carbon atom of the phenyl ring which is linked to the imino group is designated 1′ throughout and the atom carrying the branched alkyl substituent in the meta position is designated 3′.

It should be expressly pointed out that the corresponding tautomers according to general formula II are also included in the invention and therefore chemical names which indicate the structure of formula I also include the corresponding alkylanilino-2-imidazolidine derivatives according to formula II and vice versa.

Some alkylphen-1′-yl-2-imidazolidines are listed hereinafter as representative examples of all the compounds which come under general formula I or formula II.

3′-isopropyl-2′-methylphen-1′-yl-2-iminoimidazolidine 1, which is preferably in the form of the free base;

3′-tert-butyl-6′-methoxyphen-1′-yl-2-iminoimidazolidine 2, which is preferably in the form of the free base;

6′-chloro-3′-isopropyl-2′-methylphen-1′-yl-2-iminoimidazolidine 3, which is preferably in the form of the free base;

4′-chloro-3′-isopropyl-2′-methylphen-1′-yl-2-iminoimidazolidine 4, which is preferably in the form of the free base;

6′-bromo-3′-isopropyl-2′-methylphen-1′-yl-2-iminoimidazolidine 5;

6′-bromo-3′-tert-butylphen-1′-yl-2-iminoimidazolidine 6;

4′-bromo-3′-isopropyl-2′-methylphen-1′-yl-2-iminoimidazolidine 2;

5′,6′-dibromo-3′-isopropyl-2′-methylphen-1′-yl-2-iminoimidazolidine 8;

5′,6′-dichloro-3′-isopropyl-2′-methylphen-1′-yl-2-iminoimidazolidine 9;

2′,6′-dichloro-3′-isopropylphen-1′-yl-2-iminoimidazolidine 10;

4′,6′-dibromo-3′-isopropyl-2′-methylphen-1′-yl-2-iminoimidazolidine 11;

4′,6′-dichloro-3′-isopropyl-2′-methylphen-1′-yl-2-iminoimidazolidine 12;

2′,5′-dichloro-3′-isopropylphen-1′-yl-2-iminoimidazolidine 13;

2′,6′-dibromo-3′-isopropylphen-1′-yl-2-iminoimidazolidine 14;

6′-bromo-5′-chloro-3′-isopropyl-2′-methylphen-1′-yl-2-iminoimidazolidine 15;

6′-bromo-4′-chloro-3′-isopropyl-2′-methylphen-1′-yl-2-iminoimidazolidine 16;

6′-bromo-2′-chloro-3′-isopropylphen-1′-yl-2-iminoimidazolidine 17;

5′-trifluoromethyl-3′-isopropyl-2′-methylphen-1′-yl-2-iminoimidazolidine 18;

4′,5′-dichloro-3′-isopropyl-2′-methylphen-1′-yl-2-iminoimidazolidine 19;

4′-bromo-2′-chloro-3′-isopropylphen-1′-yl-2-iminoimidazolidine 20;

4′-bromo-6′-chloro-3′-isopropyl-2′-methylphen-1′-yl-2-iminoimidazolidine 21;

4′-bromo-5′-chloro-3′-isopropyl-2′-methylphen-1′-yl-2-iminoimidazolidine 22;

4′,5′-dibromo-3′-isopropyl-2′-methylphen-1′-yl-2-iminoimidazolidine 23;

2′,4′-dichloro-3′-isopropylphen-1′-yl-2-iminoimidazolidine 24;

5′-bromo-2′-methyl-3′-isopropylphen-1′-yl-2-iminoimidazolidine 25;

5′-bromo-4′-chloro-3′-isopropyl-2′-methylphen-1′-yl-2-iminoimidazolidine 6;

5′-chloro-3′-isopropyl-2′-methylphen-1′-yl-2-iminoimidazolidine 27;

5′-chloro-4′-fluoro-3′-isopropyl-2′-methylphen-1′-yl-2-iminoimidazolidine 28;

6′-trifluoromethyl-3′-isopropyl-2′-methylphen-1′-yl-2-iminoimidazolidine 29;

2′, 5′,6′-tribromo-3′-tert-butylphen-1′-yl-2-iminoimidazolidine 30;

5′,6′-dibromo-3′-tert-butylphen-1′-yl-2-iminoimidazolidine 31;

5′,6′-dibromo-3′-tert-butyl 2′-chlorophen-1′-yl-2-iminoimidazolidine 32;

2′,5′-dibromo-3′-tert-butyl 6′-methoxyphen-1′-yl-2-iminoimidazolidine 33;

2′,5′-dichloro-3′-tert-butyl-6′-methoxyphen-1′-yl-2-iminoimidazolidine 34;

2′,6′-dibromo-3′-tert-butyl 5′-chlorophen-1′-yl-2-iminoimidazolidine 35;

2′,6′-dibromo-3′-tert-butylphen-1′-yl-2-iminoimidazolidine 36;

6′-bromo-3′-tert-butyl-2′,5′-dichlorophen-1′-yl-2-iminoimidazolidine 37;

6′-bromo-3′-tert-butyl-5′-chlorophen-1′-yl-2-iminoimidazolidine 38;

2′-bromo-3′-tert-butyl-5′-chloro-6′-methoxyphen-1′-yl-2-iminoimidazolidine 39;

2′-bromo-3′-tert-butyl-6′-chlorophen-1′-yl-2-iminoimidazolidine 40;

2′-bromo-3′-tert-butyl-6′-methoxyphen-1′-yl-2-iminoimidazolidine 41;

2′-chloro-3′-tert-butyl-6′-methoxyphen-1′-yl-2-iminoimidazolidine 42;

5′-bromo-3′-tert-butyl-6′-methoxyphen-1′-yl-2-iminoimidazolidine 43;

5′-chloro-3′-tert-butyl-6′-methoxyphen-1′-yl-2-iminoimidazolidine 44;

5′-bromo-3′-tert-butyl-2′-chloro-6′-methoxyphen-1′-yl-2-iminoimidazolidine 45.

These compounds correspond to the structures:

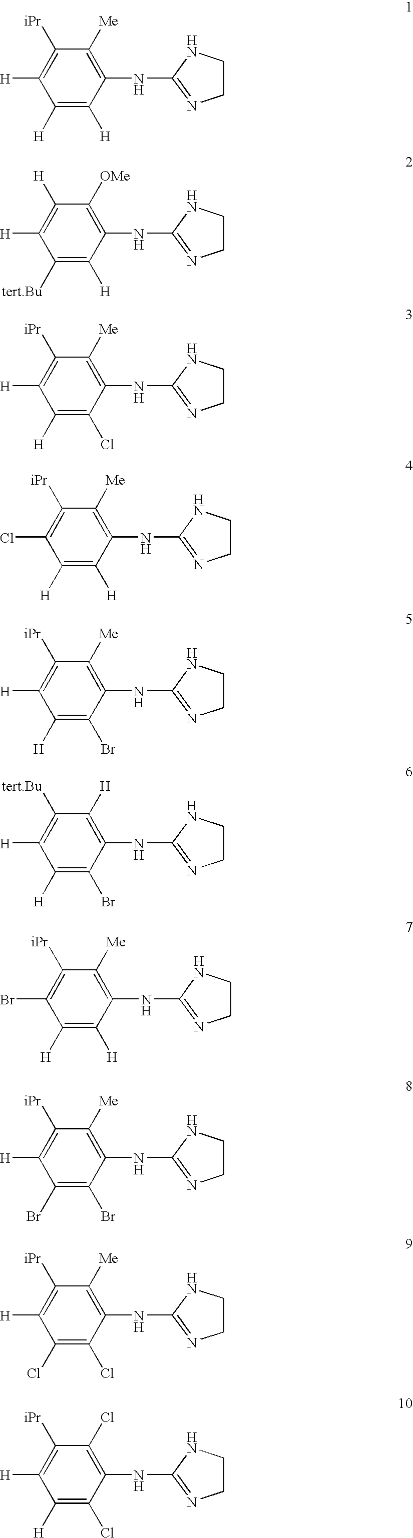

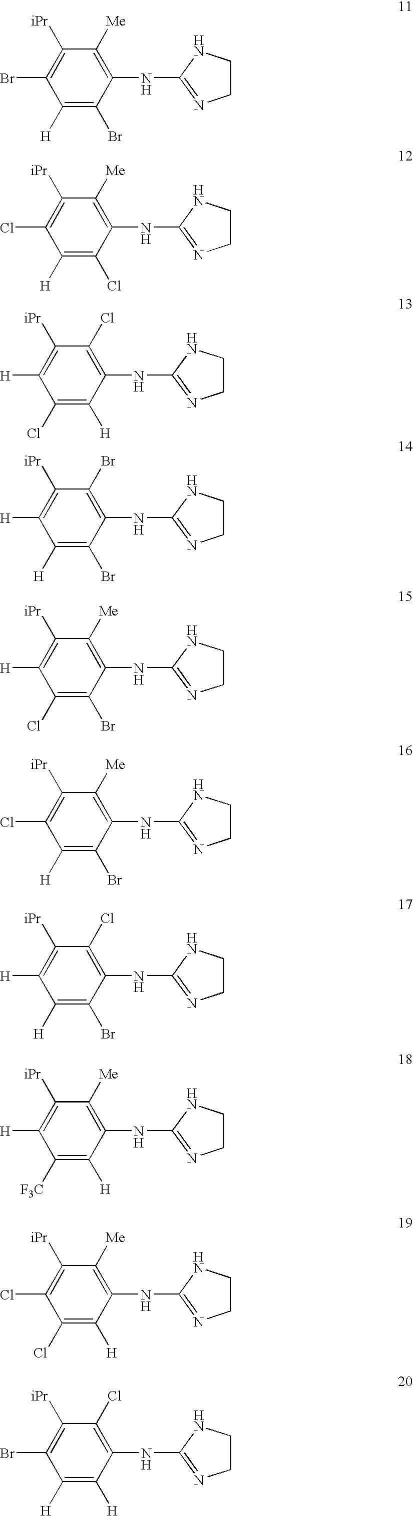

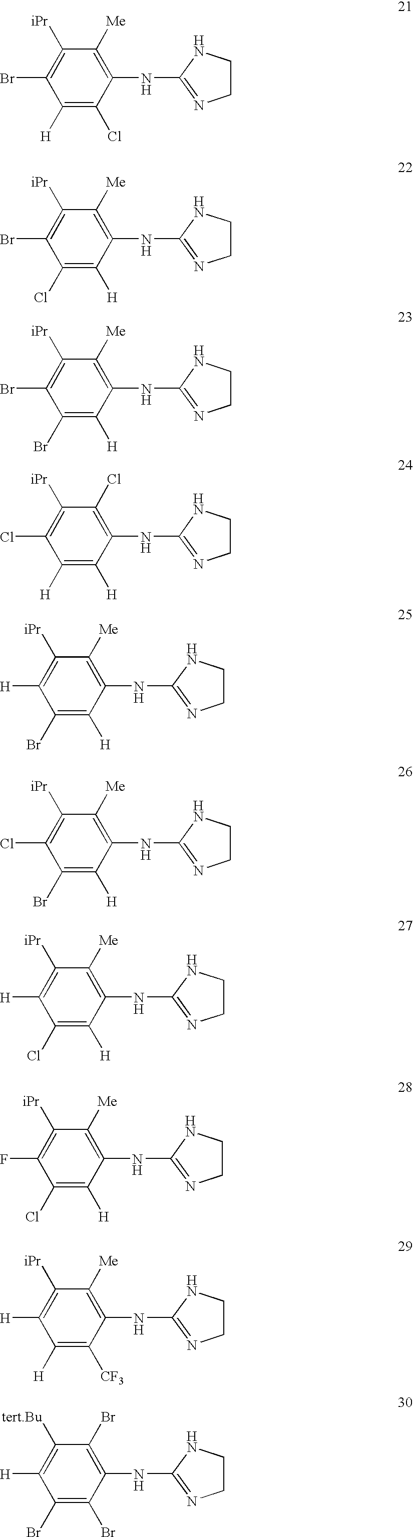

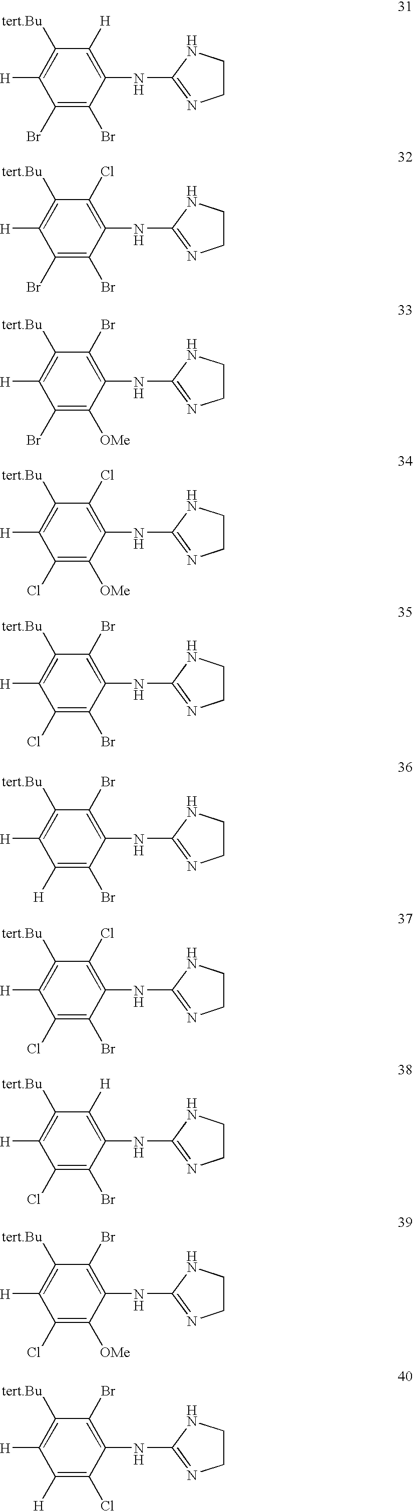

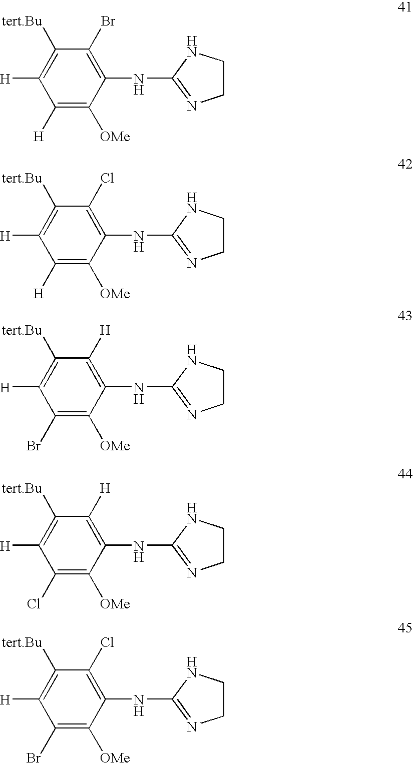

Of these compounds, the following are preferred:

3′-isopropyl-2′-methylphen-1′-yl-2-iminoimidazolidine 1;

3′-tert-butyl-6′-methoxyphen-1′-yl-2-iminoimidazolidine 2;

6′-chloro-3′-isopropyl-2′-methylphen-1′-yl-2-iminoimidazolidine 3;

4′-chloro-3′-isopropyl-2′-methylphen-1′-yl-2-iminoimidazolidine 4;

6′-bromo-3′-isopropyl-2′-methylphen-1′-yl-2-iminoimidazolidine 5;

6′-bromo-3′-tert-butylphen-1′-yl-2-iminoimidazolidine 6; and

4′-bromo-3′-isopropyl-2′-methyl-1′-phen-yl-2-iminoimidazolidine 7.

Of these compounds, the following are particularly preferred:

3′-isopropyl-2′-methylphen-1′-yl-2-iminoimidazolidine 1;

3′-tert-butyl-6′-methoxyphen-1′-yl-2-iminoimidazolidine 2;

6′-chloro-3′-isopropyl-2′-methylphen-1′-yl-2-iminoimidazolidine 3;

4′-chloro-3′-isopropyl-2′-methylphen-1′-yl-2-iminoimidazolidine 4; and

4′-bromo-3′-isopropyl-2′-methylphen-1′-yl-2-iminoimidazolidine 7.

Of these compounds, the following are most preferred:

3′-isopropyl-2′-methylphen-1′-yl-2-iminoimidazolidine 1;

3′-tert-butyl-6′-methoxyphen-1′-yl-2-iminoimidazolidine 2;

6′-chloro-3′-isopropyl-2′-methylphen-1′-yl-2-iminoimidazolidine 3; and

4′-bromo-3′-isopropyl-2′-methylphen-1′-yl-2-iminoimidazolidine 1.

The following are also preferred compounds:

4′,5′-dichloro-3′-isopropyl-2′-methylphen-1′-yl-2-iminoimidazolidine 19;

2′-chloro-3′-tert-butyl-6′-methoxyphen-1′-yl-2-iminoimidazolidine 42; and

5′-chloro-3′-isopropyl-2′-methylphen-1′-yl-2-iminoimidazolidine 27.

Examples 1, 3, 4, 5, and 7 to 29 relate to structures with an isopropyl group in the meta position, Examples 2, 6, and 30 to 45 relate to structures with a tert-butyl group in the meta position, which in turn form preferred groups.

Not only the abovementioned compounds but also the pharmaceutically acceptable acid addition salts thereof are claimed in the present invention. Suitable acids for this purpose may be either inorganic or organic by nature. Examples of suitable acids include: hydrochloric acid, hydrobromic acid, sulfuric acid, phosphoric acid, fumaric acid, citric acid, lactic acid, acetic acid, propionic acid, malic acid, succinic acid, amino acid, particularly glutamino acid or aspartic acid, carbohydrate acids and acids derived from carbohydrates. These salts may be important both for galenic preparation, they may increase the stability, especially the long-term stability of the compound and/or lead to an increase in the bioavailability. Hydrochloride salts are preferred, either the monohydrochlorides or dihydrochlorides, depending on the compound. The same is true of the preferred compounds.

As already indicated hereinbefore, the abovementioned compounds according to the invention are characterized not only by their efficacy but in particular by their pharmacological properties with regard to bioavailability and/or metabolism. It goes without saying that the most preferred compounds are those which exhibit high efficacy and bioavailability with minimal metabolic breakdown. Another significant feature for choosing particularly suitable compounds for the treatment of urinary incontinence is the selectivity with which the compound in question acts on bladder function without seriously affecting other body functions, particularly the cardiac circulatory system.

Apart from the abovementioned compounds and the pharmacologically acceptable acid addition salts thereof, the present invention also includes the use thereof for preparing medicaments and pharmaceutical preparations. These preparations include all formulations which are suitable for medical use. These include, for example, solutions, suspensions, aerosols, powders, plain and coated tablets, suppositories, creams, etc.

The compounds according to the invention, the pharmacologically acceptable acid addition salts thereof and/or pharmaceutical preparations containing them can be used medically for the treatment of complaints, particularly bladder complaints, especially urinary incontinence. The compounds according to the invention are most preferably used to treat stress incontinence.

In another aspect the present invention relates to processes for preparing the abovementioned compounds, the pharmacologically acceptable acid addition salts thereof and/or pharmaceutical preparations, as well as the use of the compound described for the preparation of other pharmacologically active derivatives thereof.

### EXAMPLES

1. Bioavailability

To determine the bioavailability, the test substances were administered orally to a group of 8 male fasted rats. As a control, animals in an identical second group were given the test substances intravenously. At specified times after administration (10 minutes, 30 minutes, 1 hour, 2 hours and 4 hours, and additionally 6 hours in the case of the animals in the oral group) 1 mL blood samples were taken from the animals in both groups. The blood samples taken in each group were mixed together (8 mL). The content of the corresponding test substances in the blood for the appropriate time was determined from the plasma after further working up by HPLC (High Performance Liquid Chromatography) using standard methods and correlated for the two groups.

RESULTS

| Compound | Bioavailability in % |
|---|---|
| 2 | 34 |
| 3 | 8 |
| 7 | 53 |

2. Metabolism

To determine the metabolism the enzyme CYP2D6 was allowed to act on the test substances. After 30 minutes, a check was made to see how much of the test substance put in had been degraded by the enzyme.

The decomposition under the effect of the enzyme HLM/60 minutes was tested analogously.

| | % Substrate Breakdown | % Substrate Breakdown |
|---|---|---|
| | After 30 Minutes Incubation | After 60 Minutes |
| Compound | with CYP2D6 | Incubation with HLM |
| 1 | 18.2 | 13.5 |
| 2 | 0.7 | 2.7 |
| 3 | 6.1 | 11.7 |
| 4 | 7.6 | 5.5 |
| 5 | 8.7 | 7.5 |
| 6 | 3.0 | 2.8 |
| 7 | 5.0 | |

3. Efficacy and Selectivity

The efficacy and selectivity of the compounds is determined as follows:

| | Activity | Activity on | Selectivity |
|---|---|---|---|
| Compound | in the Dog | Human Urethra | in the Dog |
| 1 | 78 | 11 | 0.7 |
| 2 | 71 | 30 | 0.6 |
| 3 | 70 | 41 | 0.7 |
| 4 | 59 | | 0.5 |
| 5 | 34 | | 0.4 |
| 6 | 28 | | |
| 7 | 70 | 21 | 0.7 |

Maximum contraction in the dog and activity on human urethra are percentages of contraction compared with noradrenaline. Selectivity in the dog is the percentage contraction of the dog's femoral artery at 10⁻⁵ M—percentage contraction of the carotid the dog at 10⁻⁵ M.

4. Preparation

Examples of Preparation

3′-Isopropyl-2′-methylphen-1′-yl-2-iminoimidazolidine 1

Step 1

10 g of 2-methyl-3-nitrobenzoic acid is placed in a mixture of 100 mL of methylene chloride and 1 mL of dimethylformamide. 8.9 g of thionyl chloride is slowly added dropwise and the mixture is refluxed. After 6 hours, the solvent is evaporated down under reduced pressure. 11.5 g of 2-methyl-3-nitrobenzoic acid chloride is obtained as an oil.

Step 2

11.7 g of diethyl malonate, 3.5 g of anhydrous magnesium dichloride, and 14.7 g of triethylamine are added successively to 70 mL of ethyl acetate and stirred for 0.5 hours at ambient temperature. After cooling to 10° C., 11.5 g of 2-methyl-3-nitrobenzoic acid chloride is slowly added dropwise. The mixture is then stirred at 60° C. After 3 hours, the methylene chloride is distilled off under reduced pressure. The residue is carefully mixed with 50 mL of water and adjusted to pH 1 with 4 N HCl solution. The reaction mixture is extracted 3 times with 3×100 mL ethyl acetate. The combined organic phases are washed once again with water, dried and evaporated down under reduced pressure. The oily residue is purified by chromatography (silica gel, eluant:cyclohexane/ethyl acetate (3/1). 18.6 g of diethyl 2-(2-methyl-3-nitrobenzoyl)malonate is obtained as a yellow oil.

Step 3

18.6 g of diethyl 2-(2-methyl-3-nitrobenzoyl)malonate is placed in 15 mL of concentrated acetic acid. 3.5 mL of distilled water and 3.5 mL of concentrated H₂SO₄ are slowly added. The mixture is refluxed. After 4 hours, it is cooled, diluted with 50 mL of water, made alkaline with 30% NaOH solution while cooling with ice and extracted with 3×100 mL of ethyl acetate. The combined organic phases are dried and evaporated down under reduced pressure. 8.3 g of 1-(2-methyl-3-nitrophenyl)ethanone is obtained as light yellow crystals.

Step 4

5 g of 1-(2-methyl-3-nitrophenyl)ethanone is dissolved in about 50 mL of methanol and hydrogenated at 20° C. and 5 bar with hydrogen using Raney nickel as catalyst. The catalyst is separated off and the filtrate is evaporated down under reduced pressure. The product is purified by chromatography (silica gel, eluant:cyclohexane/ethyl acetate (3/1)). 3.5 g of 1-(2-methyl-3-aminophenyl)ethanone is obtained as light yellow crystals.

Step 5

0.9 g of 60% sodium hydride oil dispersion are placed in 11.5.mL of DMSO. The suspension is stirred at 80° C. until no further development of gas can be seen (about 30 min.). It is cooled to 10° C. and then a solution of 3.5 g of 1-(2-methyl-3-aminophenyl)-ethanone and 1 mL of DMSO is slowly added dropwise. The reaction temperature rises to 45° C. After 10 minutes, a solution of 8.2 g of methyltriphenylphosphonium bromide in 23 mL of DMSO is slowly added dropwise at about 30° C. The mixture is stirred for another 4 hours without any external heat supply (RT) and then combined with 100 mL of methylene chloride and 100 mL of water. The aqueous phase is separated off and extracted twice more with 75 mL of methylene chloride. The combined organic phases are washed with water, dried and concentrated under reduced pressure. The product is purified by chromatography (silica gel, eluant:cyclohexane/ethyl acetate (3/1)). 3.4 g of 3-isopropenyl-2-methylaniline is obtained as a clear yellow oil.

Step 6

15.4 g of 3-isopropenyl-2-methylaniline is dissolved in about 150 mL of methanol and hydrogenated with hydrogen at 60° C. and 12 bar using Raney nickel as catalyst. The catalyst is separated off and the filtrate is evaporated down under reduced pressure. 14.4 g of 3-isopropyl-1-methylaniline is obtained as a clear colorless oil.

Step 7

7.45 g of 3-isopropyl-2-methylaniline and 8.7 g of N-acetylmethylmercaptoimidazolidine are refluxed for 3 hours in 100 mL of isopropanol. The solvent is distilled off under reduced pressure and the oily residue is refluxed in 100 mL of methanol. After 12 hours, the solution is concentrated down to a small volume under reduced pressure, made alkaline with 30% NaOH solution while cooling with ice and extracted twice with ethyl acetate. The combined organic phases are dried and evaporated down under reduced pressure. The oily residue is dissolved in 55 mL of HCl solution (1 N) and 55 mL of water. The solution formed is fractionally precipitated with NaOH solution (2 N) at increasing pH and extracted with diethylether until it contains the desired substance. After it has been made alkaline with NaOH solution, a solid substance is precipitated. It is suction filtered, washed with water, and dried. 6 g of 3′-isopropyl-2′-methylphen-1′-yl-2-iminoimidazolidine is obtained as a white powder, melting point 133° C.-135° C.

6′-bromo-3′-isopropyl-2′-methylphen-1′-yl-2-iminoimidazolidine 5 and

4′-bromo-3′-isopropyl-2′-methyl-1′-phen-yl-2-iminoimidazolidine 7

Step 1

7.45 g of 3-isopropyl-2-methylaniline (Example 1, Step 6) is dissolved in 75 mL of dimethylformamide and cooled to 5° C. A solution of 8.9 g of N-bromosuccinimide and 50 mL of DMF is carefully added dropwise within 45 minutes. The mixture is stirred for 1 hour at 3° C.-5° C. Then the solution is added to about 1 L of ice water and extracted three times with ethyl acetate. The combined organic phases are dried and evaporated down under reduced pressure. The two bromine isomers are isolated by chromatography (silica gel, eluant:toluene/acetone (9/1)). 1.87 g of 2′-bromo-5′-isopropyl-6′-methylaniline and 8 g of 4′-bromo-3′-isopropyl-2′-methylaniline are obtained as oils.

Step 2

2.28 g of a mixed fraction of 6′-bromo-3′-isopropyl-2′-methylaniline and 4′-bromo-3′-isopropyl-2′-methylaniline are dissolved in 45 mL of acetonitrile. 1.67 g of 2-N-acetylimidazolidin-2-one and 4.6 g of POCl₃ are added one after the other. The mixture is refluxed for 4 hours and then stirred at ambient temperature. After 12 hours, the solvent is distilled off under reduced pressure. The residue is treated with 50 mL of ice water and extracted twice with ethyl acetate. The aqueous phase is made alkaline with a NH₄OH solution while cooling. 0.5 g of a white solid is precipitated, which is suction filtered and dried (F1). The combined organic phases are evaporated down under reduced pressure. The residue is treated with water. The aqueous phase is filtered and again made alkaline with an NH₄OH solution while being cooled and extracted twice with ethyl acetate. The combined organic phases are dried and evaporated down under reduced pressure (F2: 0.9 g). F1 and F2 are refluxed together in 20 mL methanol. After 5 hours, the solvent is distilled off under reduced pressure. The two bromine isomers are separated by chromatography (silica gel, eluant:methylene chloride/methanol/ammonia (9/1/1%)). 0.15 g of 6′-bromo-3′-isopropyl-2′-methylphen-1′-yl-2-iminoimidazolidine is obtained as a hygroscopic white powder. 0.45 g of 4′-bromo-3′-isopropyl-2′-methyl-1′-phen-yl-2-iminoimidazolidine is obtained as a white powder, melting point 178° C.-181° C.

6′-chloro-3′-isopropyl-2′-methylphen-1′-yl-2-iminoimidazolidine 3 and

4′-chloro-3′-isopropyl-2′-methylphen-1′-yl-2-iminoimidazolidine 4

Step 1

7.45 g of 3-isopropyl-2-methylaniline is dissolved in 75 mL of dimethyl formamide. A solution of 6.7 g of N-chlorosuccinimide in 50 mL of DMF is slowly added dropwise. The reaction is slightly exothermic. After one night at ambient temperature the solution is diluted with water and extracted with ethyl acetate. The combined organic phases are dried and evaporated down under reduced pressure. The two chlorine isomers are isolated by chromatography (silica gel, eluant:petroleum ether/ethyl acetate (95/5)). 2.16 g of 6′-chloro-3′-isopropyl-2′-methylaniline and 3.95 g of 4′-chloro-3′-isopropyl-2′-methylaniline are obtained in the form of oils.

Step 2

In the case of the 6′-chloro-3′-isopropyl-2′-methylaniline regioisomer, the iminoimidazolidine is prepared as for compound 1, Step 7. 3.03 g of 6′-chloro-3′-isopropyl-2′-methylphen-1′-yl-2-iminoimidazolidine 3 is obtained as a hygroscopic brown powder from 3.95 g of 6′-chloro-3′-isopropyl-2′-methylaniline.

In the case of the 4′-chloro-3′-isopropyl-2′-methylaniline regioisomer, the iminoimidazolidine is prepared analogously to compounds 5 and 7, Step 2. 1.45 g of 4′-chloro-3′-isopropyl-2′-methylphen-1′-yl-2-iminoimidazolidine 4 is obtained as a hygroscopic brown powder from 2.16 g of 4′-chloro-3′-isopropyl-2′-methylaniline.

3′-tert-Butyl-6′-methoxyphen-1′-yl-2-iminoimidazolidine 2

Step 1

2.04 g of potassium isothiocyanate is dissolved at 10° C. in 60 mL of acetone. 2.38 mL of benzoyl chloride are carefully added dropwise. The white suspension is refluxed for 10 minutes and cooled at 10° C. Then a solution of 40 mL of acetone and 3.63 mL of 5-tert-butyl-2-methoxyaniline is added. The solution is then refluxed. After 3 hours, 100 mL of ice water is added and the mixture is extracted with 3×80 mL of ethyl acetate. The combined organic phases are dried and evaporated down under reduced pressure. About 9 g of brown substance is obtained which are refluxed with 11.1 mL of 50% aqueous KOH solution in 43 mL of ethanol. After 1 hour the solution is cooled and combined with 40 mL of water. The alcohol is distilled off under reduced pressure. The brown solution is buffered with 40 mL of saturated NH₄Cl solution. The product precipitated is suction filtered and dried. 9.5 g of (3-tert-butyl-6-methoxyphenyl)thiourea is obtained as a brown powder.

Step 2

9.5 g of (5-tert-butyl-2-methoxyphenyl)thiourea is dissolved in 130 mL of methanol. 1.9 mL of methyl iodide is added and the mixture is stirred at ambient temperature. After 2 hours, the solvent is distilled off under reduced pressure. The oily residue is dissolved in 1.48 mL of ethylenediamine and 100 mL of acetonitrile and the solution is heated to 105° C. After 12 hours the solvent is distilled off under reduced pressure. The residue is dissolved in 20 mL of HCl (1M) and 20 mL of water and extracted with 2×40 mL of ethyl acetate. The organic phases are separated off. The aqueous phase is neutralized with 12 mL of Na⁶H (1M) and extracted with 2×40 mL of ethyl acetate. The organic phases are separated again. The aqueous phase is made basic with 10 mL of NaOH (1M) and extracted with 2×40 mL of ethyl acetate. All 3 organic phases are combined, dried, and evaporated down under reduced pressure. The product is isolated by chromatography (silica gel, eluant:methylene chloride/methanol/ammonia (9/1/1%)). 0.85 g of 3′-tert-butyl-6′-methoxyphen-1′-yl-2-iminoimidazolidine is obtained as a white powder, melting point 172° C.-174° C.

6′-bromo-3′-tert-butylphen-1′-yl-2-iminoimidazolidine 6

Step 1

0.85 g of 3′-tert-butylphen-1′-yl-2-iminoimidazolidine is obtained as a white powder from 2.5 g of 3′-tert-butylaniline using the same method of synthesis as described for Compound 2 (Step 1 and Step 2).

Step 2

0.85 g of 3′-tert-butylphen-1′-yl-2-iminoimidazolidine is dissolved in 20 mL of methylene chloride and acidified with a few drops of glacial acetic acid. 0.2 mL of bromine are then carefully added dropwise while cooling with an ice bath. The solution is decolorized. It is stirred for 1 hour at 0° C.-5° C. Then the reaction mixture is made alkaline with concentrated NH₄OH solution. The organic phase is separated off, dried, and evaporated down under reduced pressure. The product is isolated by chromatography (silica gel, eluant:methylene chloride/methanol/ammonia (9/1/1%)). 0.28 g of 6′-bromo-3′-tert-butylphen-1′-yl-2-iminoimidazolidine is obtained as a white powder, melting point 152° C.-154° C.

5′-chloro-3′-isopropyl-2′-methylphen-1′-yl-2-iminoimidazoline 27

Step 1

14.9 g of 3-isopropyl-2-methylaniline is dissolved in 100 mL of tetrahydrofuran. 9.55 mL of acetic anhydride is added with stirring. After refluxing for one hour, the reaction mixture is cooled and petroleum ether is added thereto. The solid substance precipitated is suction filtered, washed with petroleum ether and dried. 17.5 g of 3-isopropyl-2-methylacetanilide is obtained in the form of white crystals.

Step 2

17.5 g of 3-isopropyl-2-methylacetanilide is dissolved in 90 mL of concentrated sulfuric acid while cooling gently with ice at 20° C.-25° C. 10.64 g of 1,3-dichloro-5,5-dimethylhydantoin is added in batches with stirring. The reaction mixture is stirred for 24 hours at ambient temperature and then added to 500 g of ice/water. The mixture is extracted twice with ethyl acetate and the combined organic phases are evaporated down under reduced pressure. The oil remaining is dissolved in 90 mL of methanol, 90 mL of tetrahydrofuran, and 90 mL of concentrated hydrochloric acid and refluxed for 4 hours. After cooling the mixture is made alkaline with concentrated ammonia solution. It is extracted twice with ethyl acetate and the combined organic phases are evaporated down under reduced pressure. After purification of the remaining oil by chromatography (silica gel, eluant:petroleum ether/ethyl acetate 9/1), 2.34 g of 5-chloro-3-isopropyl-2-methylaniline is obtained in the form of an oil.

Step 3-4

1.8 g of 5-chloro-3-isopropyl-2-methylaniline is reacted analogously to Example 2 via the corresponding thiourea to obtain 1.4 g of 5′-chloro-3′-isopropyl-2′-methylphen-1′-yl-2-iminoimidazoline. The product is a solid substance with a melting point of 141° C.-143° C.

The other compounds mentioned may be prepared analogously and/or according to the prior art.

1. A compound of formula I or of tautomeric formula II 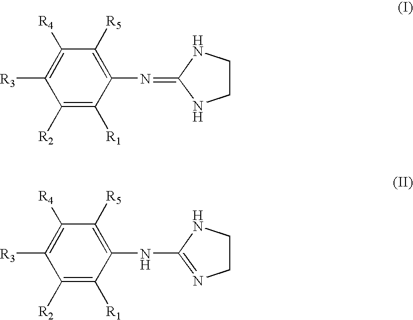
wherein:
R₁ and R₅ are each independently H, F, Cl, Br, CH₂F, CHF₂, CF₃, Me, or OMe;
R₂ and R₄ are each independently H, a branched C₃₋₆-alkyl, F, Cl, Br, CH₂F, CHF₂, CF₃ or Me, wherein at least one of the groups R₂ or R₄ is a branched C₃₋₆-alkyl; and
R₃ is H, F, Cl, Br, CH₂F, CHF₂, CF₃ or Me,
or a pharmacologically acceptable salt thereof.

2. The compound according to claim 1, wherein:
R₁ and R₅ are each independently H, F, Cl, Br, CF₃, Me, or OMe;
R₂ and R₄ are each independently H, iPr, tert-Bu, F, Cl, Br, CF₃ or Me, wherein at least one of the groups R₂ or R₄ is iPr or tert-Bu; and
R₃ is H, F, Cl, Br, CF₃ or Me.

3. The compound according to claim 1, wherein:
R₁ and R₅ are each independently H, F, Cl, Br, CF₃, Me, or OMe;
R₂ and R₄ are each independently H, iPr, tert-Bu or Me, wherein at least one of the groups
R₂ or R₄ is iPr or tert-Bu; and
R₃ is H, F, Cl, Br, or Me.

4. The compound according to claim 1, wherein:
R₁ is H, Cl, Br, or Me;
R₂ is iPr or tert-Bu;
R₃ is H, Br, or Cl;
R₄is H; and
R₅ is H, Cl, Br, or OMe.

5. The compound according to claim 1, wherein:
R₁ is H or Me;
R₂ is iPr or tert-Bu;
R₃ is H, Cl, or Br;
R₄is H; and
R₅is H or OMe.

6. The compound according to claim 1, wherein:
R₁ is H, F, Cl, Br, CH₂F, CHF₂, CF₃, or Me;
R₂ is iPr or tert-Bu; and
R₄is H.

7. A compound selected from the group consisting of:
(a) 3′-isopropyl-2′-methylphen-1′-yl-2-iminoimidazolidine 1;
(b) 3′-tert-butyl-6′-methoxyphen-1′-yl-2-iminoimidazolidine 2;
(c) 6′-chloro-3′-isopropyl-2′-methylphen-1′-yl-2-iminoimidazolidine 3;
(d) 4′-chloro-3′-isopropyl-2′-methylphen-1′-yl-2-iminoimidazolidine 4;
(e) 6′-bromo-3′-isopropyl-2′-methylphen-1′-yl-2-iminoimidazolidine 5;
(f) 6′-bromo-3′-tert-butylphen-1′-yl-2-iminoimidazolidine 6; and
(g) 4′-bromo-3′-isopropyl-2′-methylphen-1′-yl-2-iminoimidazolidine 7.

8. 5′-chloro-3′-isopropyl-2′-methylphen-1′-yl-2-iminoimidazoline 27.

9. The compound according to claim 1, wherein the compound according to formula I is an iminoimidazolidine.

10. The compound according to claim 1, wherein the compound according to formula II is an aminoimidazoline.

11. A pharmaceutical composition comprising the compound according to claim 1 and a pharmaceutically acceptable excipient.

12. A pharmaceutical composition comprising the compound according to claim 2 and a pharmaceutically acceptable excipient.

13. A pharmaceutical composition comprising the compound according to claim 3 and a pharmaceutically acceptable excipient.

14. A pharmaceutical composition comprising the compound according to claim 4 and a pharmaceutically acceptable excipient.

15. A pharmaceutical composition comprising the compound according to claim 5 and a pharmaceutically acceptable excipient.

16. A pharmaceutical composition comprising the compound according to claim 6 and a pharmaceutically acceptable excipient.

17. A pharmaceutical composition comprising the compound according to claim 7 and a pharmaceutically acceptable excipient.

18. A pharmaceutical composition comprising the compound according to claim 8 and a pharmaceutically acceptable excipient.

19. A pharmaceutical composition comprising the compound according to claim 9 and a pharmaceutically acceptable excipient.

20. A pharmaceutical composition comprising the compound according to claim 10 and a pharmaceutically acceptable excipient.

21. A method of treating urinary incontinence or a bladder disease in a patient in need thereof, the method comprising administering to the patient an effective amount of the compound according to claim 1.

22. A method of treating urinary incontinence or a bladder disease in a patient in need thereof, the method comprising administering to the patient an effective amount of the compound according to claim 2.

23. A method of treating urinary incontinence or a bladder disease in a patient in need thereof, the method comprising administering to the patient an effective amount of the compound according to claim 3.

24. A method of treating urinary incontinence or a bladder disease in a patient in need thereof, the method comprising administering to the patient an effective amount of the compound according to claim 4.

25. A method of treating urinary incontinence or a bladder disease in a patient in need thereof, the method comprising administering to the patient an effective amount of the compound according to claim 5.

26. A method of treating urinary incontinence or a bladder disease in a patient in need thereof, the method comprising administering to the patient an effective amount of the compound according to claim 6.

27. A method of treating urinary incontinence or a bladder disease in a patient in need thereof, the method comprising administering to the patient an effective amount of the compound according to claim 7.

28. A method of treating urinary incontinence or a bladder disease in a patient in need thereof, the method comprising administering to the patient an effective amount of the compound according to claim 8.

29. A method of treating urinary incontinence or a bladder disease in a patient in need thereof, the method comprising administering to the patient an effective amount of the compound according to claim 9.

30. A method of treating urinary incontinence or a bladder disease in a patient in need thereof, the method comprising administering to the patient an effective amount of the compound according to claim 10.
